(19) United States
(12) Patent Application Publication
(10) Pub. No.: US 2026/0034318 A1
(43) Pub. Date: Feb. 5, 2026

(54) MIST INHALATION POD FOR DELIVERING A THERAPEUTIC

(71) Applicant: SHAHEEN INNOVATIONS HOLDING LIMITED, Abu Dhabi (AE)

(72) Inventors: Mohammed Alshaiba Saleh Ghannam Almazrouei, Abu Dhabi (AE); Dinil Divakaran, Abu Dhabi (AE); Clement Lamoureux, Abu Dhabi (AE); Nveed Chaudhary, Abu Dhabi (AE); Peter Lomas, Wirral (GB)

(21) Appl. No.: 19/254,926

(22) Filed: Jun. 30, 2025

Related U.S. Application Data
(60) Provisional application No. 63/679,375, filed on Aug. 5, 2024.

(51) Int. Cl. A61M 15/00

(52) U.S. Cl. CPC A61M 15/001; A61M 15/0021; A61M 15/0086; A61M 2202/0007; A61M 2205/75

(57) ABSTRACT

A mist inhaler pod for use with a driver. The pod comprises a housing, a liquid barrier wall positioned within the housing having at least one liquid channel, and a liquid chamber defined by the liquid barrier wall and the housing. The pod further includes a spacer positioned within the housing, and a fluid flow manifold provided within the spacer. A sonication chamber is included within a cavity of the fluid flow manifold. An ultrasonic transducer is in communication with the sonication chamber, and a capillary conducts liquid from the liquid chamber to the sonication chamber. An air inlet conduit forms an air-tight channel for conducting air form an air inlet to the sonication chamber, and a mist outlet conduit conducts mist from the sonication chamber to the mist outlet port.

### CROSS-REFERENCE TO RELATED APPLICATION

This application claims the benefit of priority to provisional patent application Ser. No. 63/679,375, filed Aug. 5, 2024, all of which is incorporated by reference herein.

### FIELD

The present invention relates to a mist inhalation pod for use with a driver. The present invention more particularly relates to a mist inhalation pod for atomising a liquid comprising a therapeutic for inhalation by a user.

### BACKGROUND

Mist inhalers are used for generating a mist or vapour for inhalation by a user. The mist may contain a drug or medicine which is inhaled by a user and absorbed into the user's blood stream.

In particular, mist inhalers or electronic vaporising inhalers are becoming popular among smokers who want to avoid the tar and other harsh chemicals associated with traditional cigarettes and who wish to satisfy the craving for nicotine. Electronic vaporising inhalers may contain liquid nicotine, which is typically a mixture of nicotine oil, a solvent, water, and often flavouring. When the user draws, or inhales, on the electronic vaporising inhaler, the liquid nicotine is drawn into a vaporiser where it is heated into a vapour. As the user draws on the electronic vaporising inhaler, the vapour containing the nicotine is inhaled. Such electronic vaporising inhalers may have medical purpose.

Electronic vaporising inhalers and other vapour inhalers typically have similar designs. Most electronic vaporising inhalers feature a liquid nicotine reservoir with an interior membrane, such as a capillary element, typically cotton, that holds the liquid nicotine so as to prevent leaking from the reservoir. Nevertheless, these cigarettes are still prone to leaking because there is no obstacle to prevent the liquid from flowing out of the membrane and into the mouthpiece. A leaking electronic vaporising inhaler is problematic for several reasons. As a first disadvantage, the liquid can leak into the electronic components, which can cause serious damage to the device. As a second disadvantage, the liquid can leak into the electronic vaporising inhaler mouthpiece, and the user may inhale the unvapourised liquid.

Electronic vaporising inhalers are also known for providing inconsistent doses between draws. The aforementioned leaking is one cause of inconsistent doses because the membrane may be oversaturated or undersaturated near the vaporiser. If the membrane is oversaturated, then the user may experience a stronger than desired dose of vapour, and if the membrane is undersaturated, then the user may experience a weaker than desired dose of vapour. Additionally, small changes in the strength of the user's draw may provide stronger or weaker doses. Inconsistent dosing, along with leaking, can lead to faster consumption of the vaping liquid.

Additionally, conventional electronic vaporising inhalers tend to rely on inducing high temperatures of a metal heating component configured to heat a liquid in the e-cigarette, thus vaporising the liquid that can be breathed in. Problems with conventional electronic vaporising inhalers may include the possibility of burning metal and subsequent breathing in of the metal along with the burnt liquid. In addition, some may not prefer the burnt smell caused by the heated liquid.

Thus, a need exists in the art for improved mist inhalers which seek to address at least some of the problems described herein.

### BRIEF DESCRIPTION OF THE INVENTION

The present invention provides a mist inhalation pod for use with a driver as claimed in claim 1, a mist inhalation device as claimed in claim 12 and as claimed in claim 20. Preferred features of the invention are provided in the dependent claims.

The various examples of this disclosure are described below and have multiple advantages and benefits over conventional vaporising inhalers. These advantages and benefits are set out in the description below.

### REPRESENTATIVE FEATURES

Representative features are set out in the following clauses, which stand alone or may be combined, in any combination, with one or more features disclosed in the text and/or drawings of the specification.
1. A mist inhalation pod for use with a driver, the pod comprising:
   a housing having a first end, an opposite second end and at least one side wall extending between the first end and the second end;
   a first end wall proximate the first end and the side wall, the first end wall closing the first end of the housing, the first end wall being provided with a mist outlet port;
   a second end wall proximate the second end and the side wall, the second end wall closing the second end of the housing;
   a liquid barrier wall positioned within the housing and spaced apart from the first end wall, the liquid barrier wall extending towards the side wall of the housing to form a liquid seal between the liquid barrier wall and the side wall, the liquid barrier wall having at least one liquid channel with a liquid inlet and a liquid outlet;
   a liquid chamber defined by the liquid barrier wall, the first end wall and the side wall of the housing, the liquid chamber containing a liquid to be atomised, the liquid comprising a therapeutic, the liquid inlet being in liquid communication with the liquid chamber for conducting the liquid through the liquid outlet;
   a spacer positioned within the housing between the liquid barrier wall and the second end wall, the spacer having a perimeter extending toward the side wall of the housing, the spacer including a hollow interior surrounded by the perimeter;
   a fluid flow manifold positioned at least partially within the hollow interior of the spacer and including a first side proximate the first end wall, and a second side proximate the second end wall, the first side of the fluid flow manifold including a channel, and the second side of the fluid flow manifold having a cavity, the cavity including a first aperture for allowing fluid flow in a first direction, and one or more further apertures for allowing fluid flow in a second direction;
   a sonication chamber including the cavity of the fluid flow manifold;
   an ultrasonic transducer positioned between the sonication chamber and the second end wall, the ultrasonic transducer having an atomisation surface adjacent to the sonication chamber and in communication with the sonication chamber;
   a capillary having a first portion at least partly superimposed on the atomisation surface of the ultrasonic transducer and a second portion adjacent to the liquid outlet of the liquid channel, wherein the second portion of the capillary covers at least a portion of the liquid outlet and is configured to conduct the liquid from the liquid outlet to the atomisation surface to generate a mist;
   an air inlet conduit, including the one or more further apertures in the fluid flow manifold, forming an air-tight channel for conducting air through the spacer and along the channel in the first side of the fluid flow manifold, the air inlet conduit extending from proximate the second end wall, through the spacer and through the fluid flow manifold to the sonication chamber, a first end of the air inlet conduit being in fluid communication with an air inlet port proximate the second end wall of the housing and a second end of the air inlet conduit being in fluid communication with the sonication chamber; and
   a mist outlet conduit, including the first aperture in the fluid flow manifold, forming an air-tight channel for conducting the mist through the liquid chamber and any liquid contained therein, the mist outlet conduit extending from the first end wall, through the liquid chamber, through the liquid barrier wall, and through the fluid flow manifold to the sonication chamber, a first end of the mist outlet conduit being in fluid communication with a mist outlet port in the first end wall of the housing and a second end of the mist outlet conduit being in fluid communication with the sonication chamber,
   wherein, in use, the capillary conducts the liquid from the liquid chamber to the atomisation surface to be atomised, and the mist generated is conducted through the mist outlet conduit to the mist outlet port.
2. The mist inhalation pod of clause 1, further comprising a mouthpiece positioned proximate the first end of the housing, the mouthpiece including a mist inhalation port in fluid communication with the mist outlet port in the housing.
3. The mist inhalation pod of clause 1 or 2, wherein the one or more further apertures include at least three apertures, the at least three apertures being spaced around the first aperture, the fluid flow in the first direction thereby being coaxial to the fluid flow in the second direction.
4. The mist inhalation pod of any one of the preceding clauses, wherein the one or more further apertures of the air inlet conduit are positioned radially inward of the first portion of the capillary, and air enters the sonication chamber transverse to the atomisation surface of the ultrasonic transducer.
5. The mist inhalation pod of clause 4, wherein the air enters the sonication chamber perpendicular to the atomisation surface of the ultrasonic transducer.
6. The mist inhalation pod of any one of the preceding clauses, wherein the liquid barrier wall has a first surface defining the liquid chamber, and a second surface coupled to the spacer, the first surface including a recess, and the liquid inlet of the liquid channel being positioned within the recess.
7. The mist inhalation pod of any one of the preceding clauses, wherein the first portion of the capillary substantially covers the atomisation surface of the ultrasonic transducer.
8. The mist inhalation pod of any one of the preceding clauses, wherein the mist outlet conduit includes a diverter, the diverter causing the mist to take a path through the mist outlet conduit not passable by liquid droplets larger than a predetermined size.
9. The mist inhalation pod of clause 8, wherein the mist outlet conduit has a first section and a second section, the first section having an internal diameter smaller than the internal diameter of the second section, and wherein the diverter comprises a plate having an internal diameter larger than the internal diameter of the first section of the mist outlet conduit and smaller than the internal diameter of the second section of the mist outlet conduit, such that the mist conducted from the sonication chamber travels along a non-linear path from the sonication chamber to the mist outlet port.
10. The mist inhalation pod of clause 9, wherein the diverter further includes a plurality of splines spaced around the internal diameter of the second section of the mist outlet conduit, the splines configured to support the plate.
11. The mist inhalation pod of any one of clauses 8 to 10, wherein the first section of the mist outlet conduit is the first aperture of the fluid flow manifold, and the second section of the mist outlet conduit is an aperture in the liquid barrier wall, the diverter being positioned within the second section of the mist outlet conduit.
12. A mist inhalation device comprising a pod and a driver, the pod including:
   a housing having a first end, an opposite second end and at least one side wall extending between the first end and the second end;
   a first end wall proximate the first end and the side wall, the first end wall closing the first end of the housing, the first end wall being provided with a mist outlet port;
   a second end wall proximate the second end and the side wall, the second end wall closing the second end of the housing;
   a liquid barrier wall positioned within the housing and spaced apart from the first end wall, the liquid barrier wall extending towards the side wall of the housing to form a liquid seal between the liquid barrier wall and the side wall, the liquid barrier wall having at least one liquid channel with a liquid inlet and a liquid outlet;
   a liquid chamber defined by the liquid barrier wall, the first end wall and the side wall of the housing, the liquid chamber containing a liquid to be atomised, the liquid comprising a therapeutic, the liquid inlet being in liquid communication with the liquid chamber for conducting the liquid through the liquid outlet;
   a spacer positioned within the housing between the liquid barrier wall and the second end wall, the spacer having a perimeter extending toward the side wall of the housing, the spacer including a hollow interior surrounded by the perimeter;
   a fluid flow manifold positioned at least partially within the hollow interior of the spacer and including a first side proximate the first end wall, and a second side proximate the second end wall, the first side of the fluid flow manifold including a channel, and the second side of the fluid flow manifold having a cavity, the cavity including a first aperture for allowing fluid flow in a first direction, and one or more further apertures for allowing fluid flow in a second direction;
   a sonication chamber including the cavity of the fluid flow manifold;
   an ultrasonic transducer positioned between the sonication chamber and the second end wall, the ultrasonic transducer having an atomisation surface adjacent to the sonication chamber and in communication with the sonication chamber;
   a capillary having a first portion at least partly superimposed on the atomisation surface of the ultrasonic transducer and a second portion adjacent to the liquid outlet of the liquid channel, wherein the second portion of the capillary covers at least a portion of the liquid outlet and is configured to conduct the liquid from the liquid outlet to the atomisation surface to generate a mist;
   an air inlet conduit, including the one or more further apertures in the fluid flow manifold, forming an air-tight channel for conducting air through the spacer and along the channel in the first side of the fluid flow manifold, the air inlet conduit extending from proximate the second end wall, through the spacer and through the fluid flow manifold to the sonication chamber, a first end of the air inlet conduit being in fluid communication with an air inlet port proximate the second end wall of the housing and a second end of the air inlet conduit being in fluid communication with the sonication chamber; and
   a mist outlet conduit, including the first aperture in the fluid flow manifold, forming an air-tight channel for conducting the mist through the liquid chamber and any liquid contained therein, the mist outlet conduit extending from the first end wall, through the liquid chamber, through the liquid barrier wall, and through the fluid flow manifold to the sonication chamber, a first end of the mist outlet conduit being in fluid communication with a mist outlet port in the first end wall of the housing and a second end of the mist outlet conduit being in fluid communication with the sonication chamber,
   wherein, in use, the capillary conducts the liquid from the liquid chamber to the atomisation surface to be atomised, and the mist generated is conducted through the mist outlet conduit to the mist outlet port, and the driver including:
      a housing having a cavity configured to receive at least part of the pod; and
      driver circuitry for supplying a drive signal to the pod.
   13. The mist inhalation device of clause 12, further comprising a mouthpiece positioned proximate the first end of the housing, the mouthpiece including a mist inhalation port in fluid communication with the mist outlet port in the housing.
   14. The mist inhalation device of clause 12 or 13, wherein the one or more further apertures include at least three apertures, the at least three apertures being spaced around the first aperture, the fluid flow in the first direction thereby being coaxial to the fluid flow in the second direction.
   15. The mist inhalation device of any one of clauses 12 to 14, wherein the one or more further apertures of the air inlet conduit are positioned radially inward of the first portion of the capillary, and air enters the sonication chamber transverse to the atomisation surface of the ultrasonic transducer.
   16. The mist inhalation device of any one of clauses 12 to 15, wherein the air enters the sonication chamber perpendicular to the atomisation surface of the ultrasonic transducer.
   17. The mist inhalation device of any one of clauses 12 to 16, wherein the liquid barrier wall has a first surface defining the liquid chamber, and a second surface coupled to the spacer, the first surface including a recess, and the liquid inlet of the liquid channel being positioned within the recess.
   18. The mist inhalation device of any one of clauses 12 to 17, wherein the first portion of the capillary substantially covers the atomisation surface of the ultrasonic transducer.
   19. The mist inhalation device of any one of clauses 12 to 18, wherein the mist outlet conduit includes a diverter, the diverter causing the mist to take a path through the mist outlet conduit not passable by liquid droplets larger than a predetermined size.
20. The mist inhalation device of clause 17, wherein the mist outlet conduit has a first section and a second section, the first section having an internal diameter smaller than the internal diameter of the second section, and wherein the diverter comprises a plate having an internal diameter larger than the internal diameter of the first section of the mist outlet conduit and smaller than the internal diameter of the second section of the mist outlet conduit, such that the mist conducted from the sonication chamber travels along a non-linear path from the sonication chamber to the mist outlet port.
21. The mist inhalation device of clause 20, wherein the diverter further includes a plurality of splines spaced around the internal diameter of the second section of the mist outlet conduit, the splines configured to support the plate.
22. The mist inhalation device of any one of clauses 19 to 21, wherein the first section of the mist outlet conduit is the first aperture of the fluid flow manifold, and the second section of the mist outlet conduit is an aperture in the liquid barrier wall, the diverter being positioned within the second section of the mist outlet conduit.
23. A mist inhalation device comprising a pod and a driver, the pod including:
   a housing having a first end, an opposite second end and at least one side wall extending between the first end and the second end;
   a first end wall proximate the first end and the side wall, the first end wall closing the first end of the housing, the first end wall being provided with a mist outlet port;
   a second end wall proximate the second end and the side wall, the second end wall closing the second end of the housing;
   a liquid barrier wall positioned within the housing and spaced apart from the first end wall, the liquid barrier wall extending towards the side wall of the housing to form a liquid seal between the liquid barrier wall and the side wall, the liquid barrier wall having at least one liquid channel with a liquid inlet and a liquid outlet;
   a liquid chamber defined by the liquid barrier wall, the first end wall and the side wall of the housing, the liquid chamber containing a liquid to be atomized, the liquid comprising a therapeutic, the liquid inlet being in liquid communication with the liquid chamber for conducting the liquid through the liquid outlet;
   a spacer positioned within the housing between the liquid barrier wall and the second end wall, the spacer having a perimeter extending toward the side wall of the housing, the spacer including a hollow interior surrounded by the perimeter;
   a fluid flow manifold positioned at least partially within the hollow interior of the spacer and including a first side proximate the first end wall, and a second side proximate the second end wall, the first side of the fluid flow manifold including a channel, and the second side of the fluid flow manifold having a cavity, the cavity including a first aperture for allowing fluid flow in a first direction, and one or more further apertures for allowing fluid flow in a second direction;
   a sonication chamber including the cavity of the fluid flow manifold;
   an ultrasonic transducer positioned between the sonication chamber and the second end wall, the ultrasonic transducer having an atomisation surface adjacent to the sonication chamber and in communication with the sonication chamber;
   a capillary having a first portion at least partly superimposed on the atomisation surface of the ultrasonic transducer and a second portion adjacent to the liquid outlet of the liquid channel, wherein the second portion of the capillary covers at least a portion of the liquid outlet and is configured to conduct the liquid from the liquid outlet to the atomisation surface to generate a mist;
   an air inlet conduit, including the one or more further apertures in the fluid flow manifold, forming an air-tight channel for conducting air through the spacer and along the channel in the first side of the fluid flow manifold, the air inlet conduit extending from proximate the second end wall, through the spacer and through the fluid flow manifold to the sonication chamber, a first end of the air inlet conduit being in fluid communication with an air inlet port proximate the second end wall of the housing and a second end of the air inlet conduit being in fluid communication with the sonication chamber; and
   a mist outlet conduit, including the first aperture in the fluid flow manifold, forming an air-tight channel for conducting the mist through the liquid chamber and any liquid contained therein, the mist outlet conduit extending from the first end wall, through the liquid chamber, through the liquid barrier wall, and through the fluid flow manifold to the sonication chamber, a first end of the mist outlet conduit being in fluid communication with a mist outlet port in the first end wall of the housing and a second end of the mist outlet conduit being in fluid communication with the sonication chamber,
   wherein, in use, the capillary conducts the liquid from the liquid chamber to the atomisation surface to be atomised, and the mist generated is conducted through the mist outlet conduit to the mist outlet port, and the driver including:
      a housing having at least one wall, and a cavity configured to receive at least a part of the pod;
      an insert positioned within the cavity, the insert having a first surface, a second surface, and a side wall extending therebetween, the insert including a duct extending from the side wall to the first surface, the duct having an opening in the side wall and an opening in the first surface; and
      a hole in the wall of the driver,
   wherein the hole in the wall of the driver aligns with the opening in the side wall of the duct, and the opening in the first surface of the duct aligns with the air inlet conduit of the pod,
   the air conducted from the surroundings to the air inlet conduit via the hole in the wall of the driver and the duct provided by the insert.

### BRIEF DESCRIPTION OF THE FIGURES

In order that the present disclosure may be more readily understood, preferable embodiments thereof will now be described, by way of example only, with reference to the accompanying drawings, in which:

FIG. 1 is a diagrammatic view of a mist inhalation pod embodying the present disclosure;

FIG. 2 is a diagrammatic cross-sectional view of the mist inhalation pod, embodying the present disclosure;

FIG. 3 is a diagrammatic cross-sectional view of a part of the mist inhalation pod, embodying the present disclosure;

FIG. 4 is a diagrammatic exploded view of a part of the mist inhalation pod, embodying the present disclosure;

FIG. 5 is a diagrammatic view of a part of the mist inhalation pod, embodying the present disclosure;

FIG. 6 is a diagrammatic view of a part of the mist inhalation pod, embodying the present disclosure;

FIG. 7 is a diagrammatic view of the part of the mist inhalation pod illustrated in FIG. 6;

FIG. 8 is a diagrammatic view of a part of the mist inhalation device, embodying the present disclosure;

FIG. 9 is a further diagrammatic view of the part of the mist inhalation device illustrated in FIG. 8;

FIG. 10 is a diagrammatic partial view of the mist inhalation device, embodying the present disclosure;

FIG. 11 is a diagrammatic cross-sectional view of the mist inhalation device, embodying the present disclosure;

FIG. 12 is a diagrammatic cross-sectional view of the mist inhalation device, embodying the present disclosure;

FIG. 13 is a diagrammatic cross-sectional view of the mist inhalation device, embodying the present disclosure; and

FIG. 14 is a diagrammatic cross-sectional view of the mist inhalation device, embodying the present disclosure.

### DETAILED DESCRIPTION OF THE DISCLOSURE

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

The following disclosure provides many different embodiments, or examples, for implementing different features of the subject matter provided. Specific examples of components, concentrations, applications and arrangements are described below to simplify the present disclosure. These are, of course, merely examples and are not intended to be limiting. For example, the attachment of a first feature and a second feature in the description that follows may include embodiments in which the first feature and the second feature are attached in direct contact, and may also include embodiments in which additional features may be positioned between the first feature and the second feature, such that the first feature and the second feature may not be in direct contact. In addition, the present disclosure may repeat reference numerals and/or letters in the various examples. This repetition is for the purpose of simplicity and clarity and does not in itself dictate a relationship between the various embodiments and/or configurations discussed.

The following disclosure describes representative arrangements or examples. Each arrangement or example may be considered to be an embodiment and any reference to an “arrangement” or an “example” may be changed to “embodiment” in the present disclosure.

Conventional electronic vaporizing inhalers tend to rely on inducing high temperatures of a metal component configured to heat a liquid in the inhaler, thus vaporizing the liquid that can be breathed in. The liquid typically contains nicotine and flavorings blended into a solution of propylene glycol (PG) and vegetable glycerin (VG), which is vaporized via a heating component at high temperatures. Problems with conventional inhalers may include the possibility of burning metal and subsequent breathing in of the metal along with the burnt liquid. In addition, some may not prefer the burnt smell or taste caused by the heated liquid.

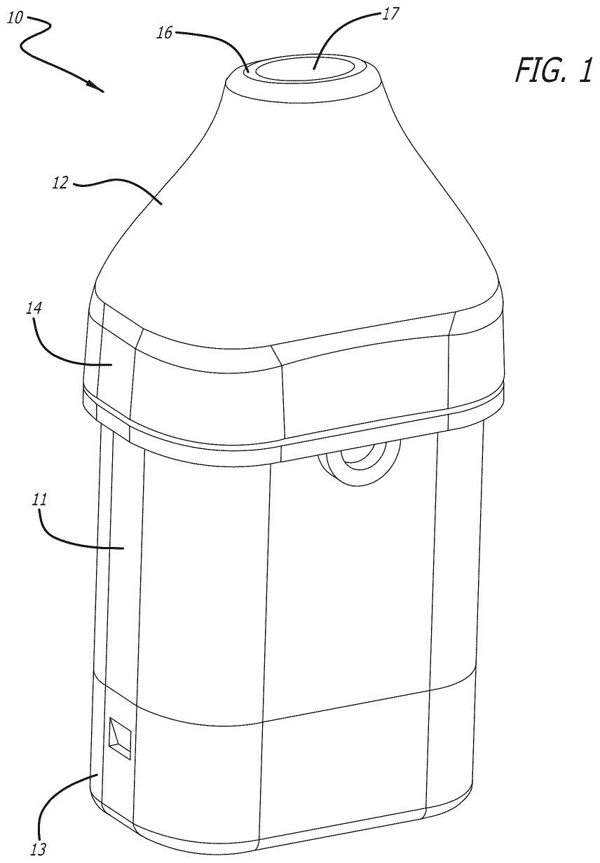

FIGS. 1 to 14 illustrate a pod and driver comprising a sonication chamber. It is noted that the expression “mist” used in the following disclosure means the liquid is not heated as usually in traditional inhalers known from the prior art. In fact, traditional inhalers use heating elements to heat the liquid above its boiling temperature to produce a vapor, which is different from a mist. A vapor involves a phase change from the liquid to a gas, whereas the liquid dispersed within the air in the present disclosure remains in the liquid phase.

When sonicating liquids at high intensities, the sound waves that propagate into the liquid media result in alternating high-pressure (compression) and low-pressure (rarefaction) cycles, at different rates depending on the frequency. During the low-pressure cycle, high-intensity ultrasonic waves create small vacuum bubbles or voids in the liquid. This phenomenon is termed cavitation. When the bubbles attain a volume at which they can no longer absorb energy, they collapse violently during a high-pressure cycle. During the implosion, very high pressures are reached locally. At cavitation, broken capillary waves are generated, and tiny droplets break the surface tension of the liquid and are quickly released into the air, taking mist form.

The following will explain more precisely the cavitation phenomenon.

When the liquid is atomized by ultrasonic vibrations, micro water bubbles are produced in the liquid.

The bubble production is a process of formation of cavities created by the negative pressure generated by intense ultrasonic waves generated by the means of ultrasonic vibrations.

High intensity ultrasonic sound waves leading to rapid growth of cavities with relatively low and negligible reduction in cavity size during the positive pressure cycle.

Ultrasound waves, like all sound waves, consist of cycles of compression and expansion. When in contact with a liquid, Compression cycles exert a positive pressure on the liquid, pushing the molecules together. Expansion cycles exert a negative pressure, pulling the molecules away from another.

Intense ultrasound waves create regions of positive pressure and negative pressure. A cavity can form and grow during the episodes of negative pressure. When the cavity attains a critical size, the cavity implodes.

The amount of negative pressure needed depends on the type and purity of the liquid. For truly pure liquids, tensile strengths are so great that available ultrasound generators cannot produce enough negative pressure to make cavities. In pure water, for instance, more than 1,000 atmospheres of negative pressure would be required, yet the most powerful ultrasound generators produce only about 50 atmospheres of negative pressure.

The tensile strength of liquids is reduced by the gas trapped within the crevices of the liquid particles. The effect is analogous to the reduction in strength that occurs from cracks in solid materials. When a crevice filled with gas is exposed to a negative-pressure cycle from a sound wave, the reduced pressure makes the gas in the crevice expand until a small bubble is released into solution.

However, a bubble irradiated with ultrasound continually absorbs energy from alternating compression and expansion cycles of the sound wave. These cause the bubbles to grow and contract, striking a dynamic balance between the void inside the bubble and the liquid outside. In some cases, ultrasonic waves will sustain a bubble that simply oscillates in size. In other cases, the average size of the bubble will increase.

Cavity growth depends on the intensity of sound. High-intensity ultrasound can expand the cavity so rapidly during the negative-pressure cycle that the cavity never has a chance to shrink during the positive-pressure cycle. In this process, cavities can grow rapidly in the course of a single cycle of sound.

For low-intensity ultrasound the size of the cavity oscillates in phase with the expansion and compression cycles. The surface of a cavity produced by low-intensity ultrasound is slightly greater during expansion cycles than during compression cycles. Since the amount of gas that diffuses in or out of the cavity depends on the surface area, diffusion into the cavity during expansion cycles will be slightly greater than diffusion out during compression cycles. For each cycle of sound, then, the cavity expands a little more than it shrinks. Over many cycles the cavities will grow slowly.

It has been noticed that the growing cavity can eventually reach a critical size where it will most efficiently absorb energy from the ultrasound. The critical size depends on the frequency of the ultrasound wave. Once a cavity has experienced a very rapid growth caused by high intensity ultrasound, it can no longer absorb energy as efficiently from the sound waves. Without this energy input the cavity can no longer sustain itself. The liquid rushes in and the cavity implodes due to a non-linear response.

The energy released from the implosion causes the liquid to be fragmented into microscopic particles which are dispersed into the air as mist.

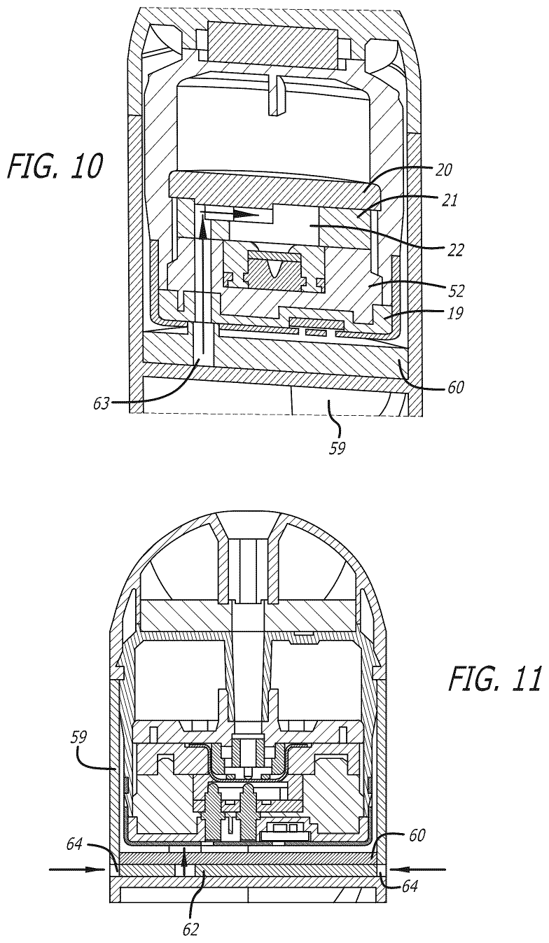

FIG. 1 shows a mist inhalation pod (hereinafter referred to as a pod) 10 according to some embodiments if the present disclosure. The pod 10 is configured to be releasably attached to a driver (shown in FIGS. 10 to 14). The driver houses the components required to store electrical energy and provide an electrical signal to the pod 10. In other examples, the pod 10 may be fixed to, formed integrally with or otherwise non-releasably attached to the driver.

The pod 10 comprises a housing 11 having a first end and an opposite second end, a mouthpiece 12, and an end cap 13. Between the two ends of the housing extends at least one side wall. In some examples, the housing 11 is of injection moulded plastic, specifically polypropylene that is typically used for medical applications. In some examples, the housing 11 is of a heterophasic copolymer. More particularly a BF970MO heterophasic copolymer, which has an optimum combination of very high stiffness and high impact strength. Parts moulded with this material also exhibit good anti-static performance.

A heterophasic copolymer such as polypropylene is particularly suitable for the pod housing 11 since this material minimises or does not cause condensation of the aerosol as it flows through the mouthpiece 12 to the user. This plastic material can also be directly recycled easily using industrial shredding and cleaning processes.

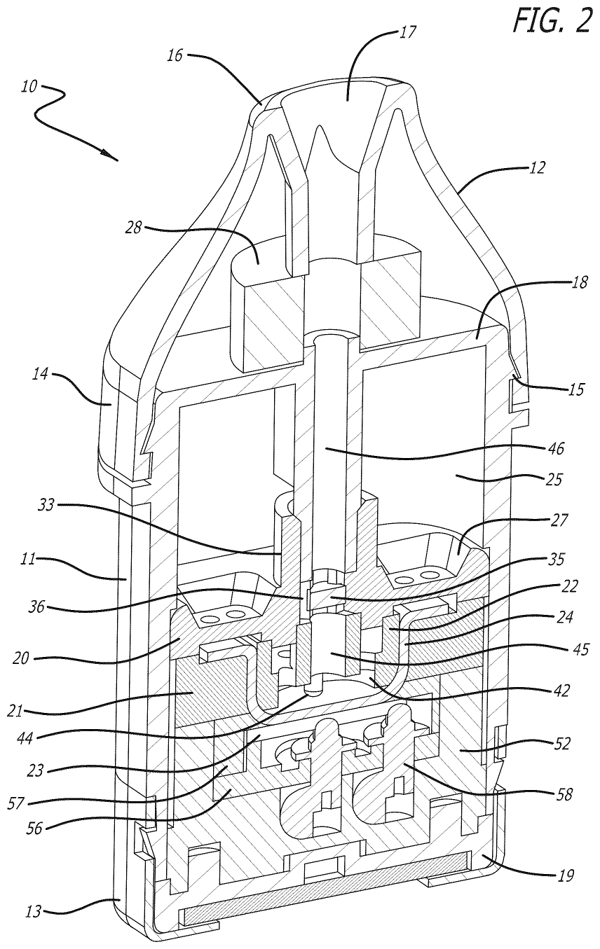

The mouthpiece 12 comprises a base 14 having an opening which receives a connector portion 15 positioned towards the first end of the housing 11, as seen in FIG. 2. The connector portion 15 may comprises at least one latch element that engages a latch recess to retain the mouthpiece 12 in connection with the housing 11.

The mouthpiece 12 narrows progressively from the base 14 to a distal end 16. The distal end 16 comprises a mouthpiece outlet port 17 to enable mist to exit the pod 10 for inhalation by the user.

The end cap 13 attaches to the second end of the housing 11. The end cap 13 comprises four walls that define a recess for receiving the second end of the housing 11. Opposing side walls of the end cap 13 each comprise a respective retainer slot which are configured to receive respective engaging portions positioned towards the second end of the housing 11. The end cap 13 comprises an aperture to allow access to components and features located at the second end of the housing 11.

In some examples, the end cap 13 is of metal. However, in other examples, the end cap 13 may be of a different material or may be omitted entirely.

FIG. 2 illustrates the internal components of the pod 10 according to some examples. The housing 11 comprises a first end wall 18 proximate the first end of the housing 11, and a second end wall 19 proximate the second end of the housing 11. The first end wall 18 and the second end wall 19 may be configured to close the first end and the second end of the housing 11, respectively.

An absorbing element 28 may be positioned in or adjacent to the mist flow path so as to absorb any liquid droplets as the mist is conducted towards the mouthpiece 12. Preferably, the absorbing element 28 is at least partly of bamboo fibre.

The pod 10 comprises a mist outlet conduit, at least a first section 46 of which may be integrally formed with the housing 11 and extend from the first end wall 18 towards the second end wall 19.

Referring now to FIGS. 2 to 7, the housing 11 may enclose a liquid barrier wall 20, a spacer 21, a fluid flow manifold 22, an ultrasonic transducer 23, and a capillary 24.

The liquid barrier wall 20 is positioned within the housing 11 and extends towards the side wall to create a seal between the liquid barrier wall 20 and the side wall of the housing 11. The liquid barrier wall 20 is spaced apart from the first end wall 18 to form a liquid chamber 25 therebetween.

The liquid chamber 25 is configured to hold a liquid to be atomised. The liquid preferably includes at least one therapeutic suitable for the aerosol delivery to the lungs through inhalation by a patient for providing a desired treatment to the patient. Some examples of therapeutics include, but are not limited to, aerosol delivery of pharmacological agents to the lungs to promote a systemic or direct clinical effect while producing a minimal adverse-effect. A therapeutic may also include for example, but not limited to, natural drugs, cannabinoid derivatives such as CBD for pain relief and other treatments; botanicals; opioids; RNA; DNA; chemotherapies; subcellular components including for example ribosomes, endoplasmic reticulum, cytoskeleton, and mitochondria; supplements for performance enhancement; albuterol/salbutamol for asthma patients; agents such as beta-lactams, polymyxins, and aminoglycosides bactericidal antibiotics; amphotericin B; morphine; fentanyl; prostacyclin, amiloride, and interferon-g; and cyclosporine as rescue therapy rejection in lung-transplantation patients and a treatment for asthma.

In some examples, the liquid chamber 218 contains an e-liquid or liquid comprising at least one cannabinoid or phytocannabinoid derived from a cannabis plant. In some examples, the at least one cannabinoid or phytocannabinoid comprises one or more of tetrahydrocannabinol (THC), cannabidiol (CBD) and/or cannabinol (CBN).

In some examples, the liquid chamber 218 contains an e-liquid or liquid comprising a psychedelic compound or compounds for use as a therapeutic. In some examples, the psychedelic compound or compounds is one or more of lysergic acid diethylamide (LSD), 3,4-methylenedioxymethamphetamine (MDMA), ketamine, esketamine, ibogaine, mescaline, a tryptamine, a substituted tryptamine, O-acetylpsilocin (4-AcO-DMT), psilocybin (4-PO-DMT), psilocin (4-HO-DMT), O-methylbufotenin (5-MeO-DMT), 20ufotenine (5-HO-DMT) and/or N,N-Dimethyltryptamine (DMT or N,N-DMT).

The devices disclosed herein are for use with any therapeutics, drugs or other compounds, with the drug or compound being provided in a liquid within the liquid chamber of the device for aerosolisation by the device. In some examples, the devices disclosed herein are for use with therapeutics, drugs and compounds including, but not limited to, the following:

### Respiratory

### Brochodilators

Olodaterol
Levalbuterol
Berodual (Ipratropium bromide/Fenoterol)
Combivent (Ipratropium bromide/Salbutamol)

### Anti-Inflammatory

Betamethasone
Dexamethasone
Methylprednisolone
Hydrocortisone

### Mucolytics

N-Acetylcysteine

### Pulmonary Hypertension

Sildenafil
Tadalafil
Epoprostenol
Treprostenil
Iloprost

### Infectious Disease

### Antimicrobials

Aminoglycosides (Gentamicin, Tobramycin, Amikacin, Colomycin, Neomycin, Liposomal Amikacin,)
Quinolones (Ciprofloxacin, Levofloxacin, Moxifloxacin Ofloxacin)
Macrolides (Azithromycin)
Minocycline
Betalactams (Piperacillin-Tazobactam, Ceftazidime Ticarcillin)
Cephalosporins (Cefotaxime, Cefepime, Ceftriaxone, Cefotaxime) Glycopeptides (Vancomycin)
Meropenem
Polymixin (Colistin, Polymixin B)

### Antifungals

Amphotericin
Fluconazole
Caspofungen

### Antivirals

Valganciclovir
Favipiravir
Remdisivir
Acyclovir
Anti TB
Isoniazid
Pyrazinamide
Rifampin

Ethambutol

### Oncology

Biologics
Gilotrif
Afatinib
Caplacizumab
Dupilumab
Isarilumab
Alirucomab
Volasertib_
Nintedanib
Imatinib
Sirolimus

### Chemotherapy

Azacitidine
Decitabine
Docetaxel
Gemcitabine
Cisplatinum

### CNS & PSYCH

Sodium valproate
Teriflunomide
Zomitriptan

### METABOLIC/HORMONAL

Insulin
Estrogen

### IMMUNOLOGY

Vaccine
Monoclonal Antibodies
Stem Cells

### Vitamins

Zinc
Ascorbic Acid

### Miscellaneous

Niclosamide
Hydroxychloroquine
Ivermectin

The liquid barrier wall 20 comprises a liquid channel 26 having a liquid inlet and a liquid outlet. The liquid channel 26 passes entirely through the liquid barrier wall 20 so as to allow liquid communication between the liquid chamber 25 and a capillary 24. The liquid barrier wall 20 may have more than one liquid channel 26 to improve the liquid flow rate to the capillary 24, or to improve the dispersion of the liquid over a larger surface area of the capillary 24.

The liquid barrier wall 20 may further comprise one or more recesses 27 in the planar face that defines the liquid chamber 25. Providing the liquid inlet of the liquid channel 26 in the recess 27 allows the liquid chamber 25 to be fully depleted of liquid before the pod 10 needs to be refilled or disposed of, due to the recess 27 representing the lowest point in the liquid chamber 25.

The liquid barrier wall 20 may comprise blind holes in its lower surface (i.e. the planar surface opposite that on which the recess 27 is provided) configured to accept pegs 31 of the spacer 21.

The liquid barrier wall 20 may further comprise a central protrusion 33 which includes a through hole 43. The central protrusion 33 and through hole 43 form a second section 47 of the mist outlet conduit. Within the bore of the through hole 43 may be a mist diverter assembly 34.

The purpose of the mist diverter assembly 34 is to prevent, or at least reduce the number of, large liquid droplets from flowing to the mouthpiece 12 and, ultimately, into the mouth of the user. The mist diverter assembly 34 may comprise a plate 35, which may be supported by a plurality of splines 36 spaced around the bore of the through hole 43.

The spacer 21 is positioned between the liquid barrier wall 20 and the second end wall 19 within the housing 11. The spacer 21 has an outer wall forming a perimeter, and a hollow interior. Once the pod 10 is assembled, the perimeter of the spacer 21 extends towards the side wall of the housing 11.

The spacer 21 includes a slot 32 which may extend axially through the perimeter portion. In some examples, the slot 32 may not extend through the top surface of the perimeter portion, the top surface being the surface which may abut the liquid barrier wall 20. The slot 32 forms a section of the air inlet conduit once the pod 10 is assembled.

The spacer 21 further includes indentations 37 for accepting at least part of the second portion of the capillary 24. The indentations 37 may align with the liquid channels 26 of the liquid barrier wall 20 and thereby permit at least part of the second portion of the capillary 24 to lie adjacent the liquid outlet of the liquid channels 26. It will be appreciated that the spacer 21 may include only one indentation 37, or need not include indentations 37 at all.

In some envisaged alternative embodiments, the spacer 21 may be spaced from the lower surface of the liquid barrier wall 20. In a further alternative, indentations may additionally or alternatively be provided in the lower surface of the liquid barrier wall 20.

At least a part of at least one of the liquid barrier wall 20, the spacer 21, and the second end wall 19 may be at least partly of a resiliently deformable material so as to prevent liquid leaking from the liquid chamber 25. Such a material may comprise silicone.

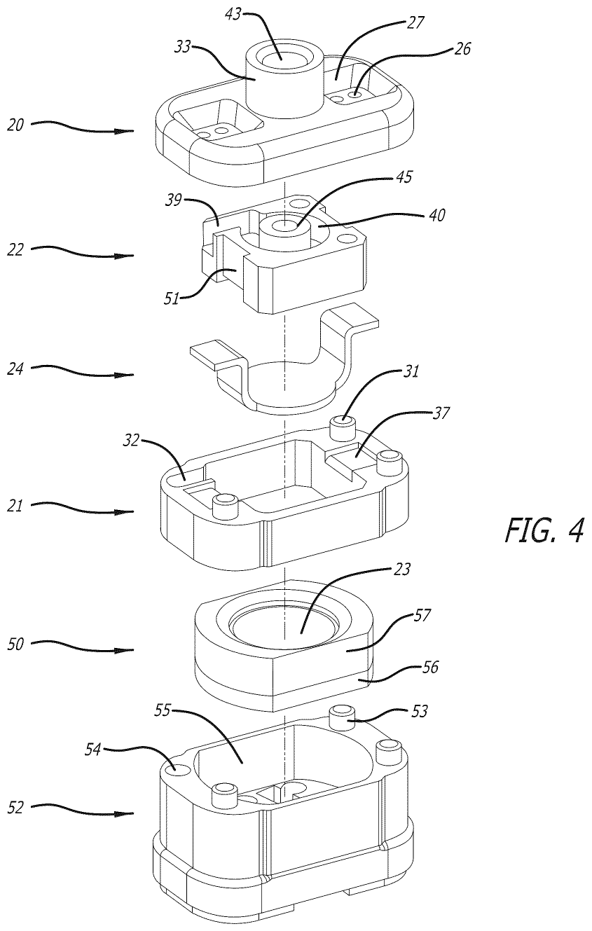

The hollow interior of the spacer 21 is sized to at least partially receive the fluid flow manifold 22. The manifold 22 has a first side and an opposite second side, the first side being the upper side as shown in FIG. 4, and the second side being the lower side shown in FIG. 7. When assembled, the first side of the manifold 22 is proximate the first end wall 18 and the second side of the manifold is proximate the second end wall 19.

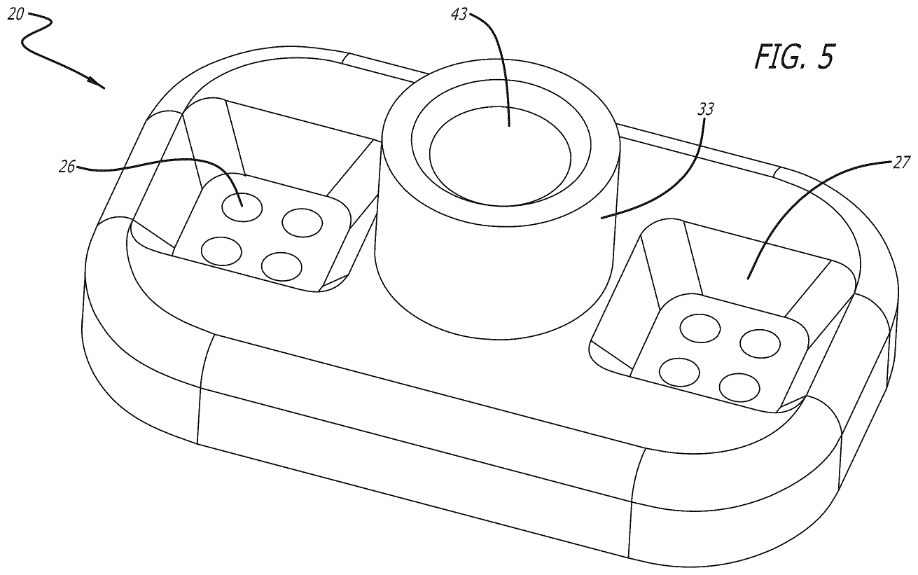

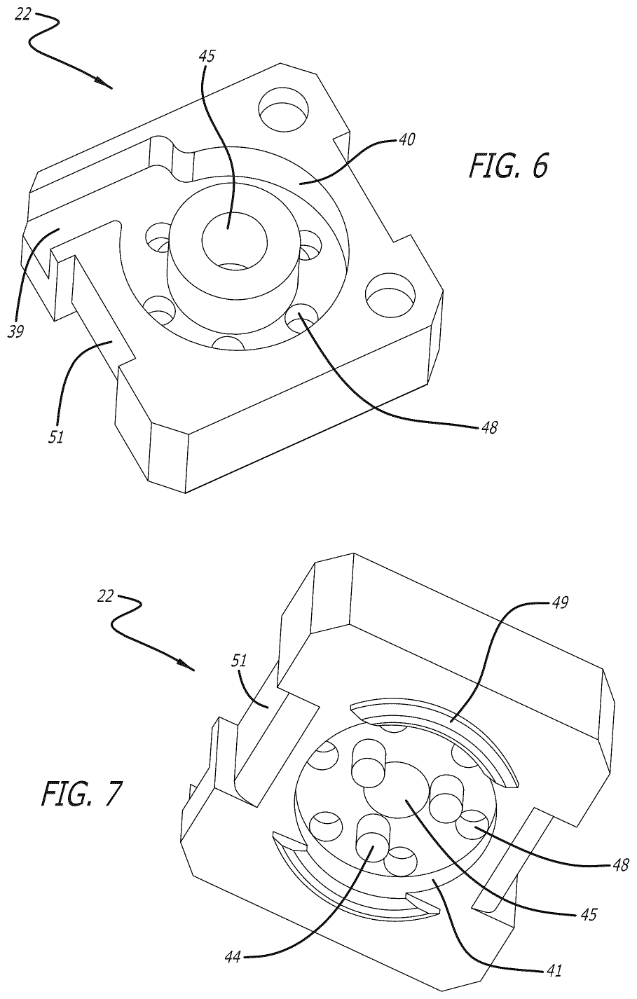

The first side of the manifold 22, shown in FIG. 6, may comprise a channel 38. The channel 38 extends from an edge of the manifold 22 which is proximate the slot 32 in the spacer 21. The channel 38 may preferably comprise a straight portion 39 and an annular portion 40. Alternatively, either or both of the straight and annular portions may be of any shape.

The second side of the manifold 22 is shown in FIG. 7. The second side of the manifold 22 includes a cavity 41. Once the pod 10 is assembled, the cavity 41 partially defines a sonication chamber 42 in which the mist is produced.

The base of the cavity 41 includes a first aperture 45. The first aperture 45 extends through the centre of the annular channel 40 in the first side of the manifold 22. The first aperture 45 and the centre of the annular channel 40 thereby form a third section of the mist outlet conduit.

The base of the cavity 41 also includes one or more further apertures 48. The further apertures 48 extend from the base of the cavity 41 through to the annular portion 40 of the channel 38 in the first side of the manifold 22. The further apertures 48 thereby allow air into the sonication chamber 42 at an angle transverse to the atomisation surface of the ultrasonic transducer 23. The airflow therefore contacts the ultrasonic transducer with an increased force and may result in more efficient aerosolization and/or mist extraction. In some examples, the air flow is substantially perpendicular to the atomisation surface.

Preferably, there are at least three further apertures 48. In examples having two or more further apertures 48, the further apertures 48 are spaced, preferably evenly spaced, around the first aperture 45. The air inlet flow and the mist outlet flow may therefore be coaxial.

The one or more further apertures of the air inlet conduit are preferably positioned radially inward of the edge of the atomisation surface.

A plurality of fingers 44 extend from the base of the cavity 41 towards the second side of the manifold 22. The fingers 44 serve as biasing elements, and are configured to urge the first portion of the capillary 24 into contact with the ultrasonic transducer 23 to enhance atomisation of the liquid. The fingers 44 may be of any size, shape and material.

The second side of the manifold 22 comprises elongate frusto-annular lips 49 around the cavity 41. The lips 49 may be of substantially the same thickness as the capillary 24 such that when the pod 10 is assembled, the lips 49 contact the transducer holder 50. The lips 49 therefore serve to prevent liquid leaking from capillary 24 between the manifold 22 and the transducer holder 50.

The gaps between the lips 49 allow the capillary 24 to extend between the liquid channel 26 of the liquid barrier wall 20 and the sonication chamber 42. In order to aid the passage of the capillary 24, the manifold 22 may include open slots 51 in its side surfaces, as illustrated in FIGS. 6 and 7. In some examples, the slots may additionally or alternatively be positioned in the wall of the hollow interior of the spacer 21. Any change in angle of a slot, indentation, or otherwise which is configured to receive a portion of the capillary 24 may include a radius so as to not interfere with the fluid flow through the capillary 24.

The pod 10 further includes a lower body portion 52 positioned between the spacer 21 and the second end wall 19. The lower body portion 52 has an upper surface, a lower surface, and at least one side extending therebetween. The lower surface may abut the second end wall 19 of the housing. The upper surface may include dowels 53 configured to locate in corresponding holes (not shown) in the lower surface of the spacer 21. In some examples, the dowels 53 are integrally formed within the lower body portion 52. A through hole 54 may extend through both the upper and lower surfaces of the lower body portion 52. The through hole 54 serves as a section of the air inlet conduit.

The lower body portion 52 comprises a cavity 55 configured to accept the transducer holder 50. At least one, and preferably a plurality of passages, extend between the base of the cavity 55 and the lower surface of the lower body portion 52, the passages serving to allow electrical connections to pass therethrough.

The transducer holder 50 is sized and shaped to be accepted by the lower body portion 52. The ultrasonic transducer 23 is supported in position adjacent to and in communication with the sonication chamber 42 by the transducer holder 50. The transducer holder 50 comprises a lower disc portion 56 and an upper annular portion 57, at least one of which may comprise a resiliently deformable material. The lower disc portion 56 may be generally planar. The lower disc portion 56 comprises holes through which electrical contacts 58 may extend to enable the transfer of a signal to the ultrasonic transducer 23. The lower disc portion 56 further comprises an annular ridge to act as the supporting surface for the underside of the ultrasonic transducer 23.

The upper annular portion 57 is sized and shaped to contact the lower disc portion 56 and the spacer 21. The upper annular portion 57 further acts to clamp the outer rim of the ultrasonic transducer 23 between itself and the annular ridge of the lower disc portion 56 such that any vibrations are efficiently transferred to the capillary 24, but preferably isolated from the housing. The upper annular portion 57 may incorporate a chamfer or radius on its inner edge, thereby aiding the change in direction of air flow within the sonication chamber 42.

The ultrasonic transducer 23 is configured to convert an electrical input signal into high frequency vibrations. The atomisation surface of the ultrasonic transducer 23 is adjacent to and in communication with the sonication chamber 42 in the form of contacting the capillary 24. In use, the atomisation surface is configured to turn the liquid, which saturates the capillary 24, into a mist.

The capillary 24 may be of any material capable of transporting liquid by capillary action. The shape of the capillary 24 may be determined by the channel formed between the manifold 22 and the spacer 21, and also between the spacer 21 and the liquid barrier wall 20. The capillary 24 comprises a first portion and a second portion. The first portion is at least partially superimposed on the atomisation surface of the ultrasonic transducer 23, and preferably substantially covers the atomisation surface of the ultrasonic transducer 23. The second portion is adjacent the liquid outlet of the liquid channel 26, and preferably covers at least a portion of the liquid outlet. More preferably, the second portion of the capillary 24 completely covers all liquid channels 26 in the liquid barrier wall 20. The liquid from the liquid chamber 25 is therefore conducted from the liquid outlet of the liquid channel 26 to the atomisation surface of the ultrasonic transducer 23 by the capillary 24.

Referring now to FIG. 4, which illustrates an exploded view of part of the pod 10 and thus assists with the visualisation of the assembly. The transducer holder 50 is placed within the cavity 55 of the lower body portion 52, and the spacer 21 is coupled to the lower body portion 52. The dowels 53 of the lower body portion 52 engage in holes on the lower surface of the spacer 21. The dowels 53 and holes may be any of a clearance, transitional, or interference fit, and serve to prevent excessive movement of the lower body portion 52 relative to the spacer 21. The through hole 54 and the slot 32 align so as to form a part of the air inlet conduit.

The capillary 24 is inserted through the hollow interior of the spacer 21 so that the first portion of the capillary 24 is superimposed on the atomisation surface of the ultrasonic transducer 23. The second portions of the capillary 24 are positioned within the indentations 37 in the spacer 21.

The manifold 22 is positioned within the hollow cavity of the spacer 21, thereby forming the sonication chamber 42. The biasing elements 44 urge the first portion of the capillary 24 into contact with the atomisation surface of the ultrasonic transducer 23. The second portion of the capillary 24 passes through a channel formed between the manifold slots 51 and the spacer 21. The straight portion 39 of the manifold channel 38 aligns with the slot 32 in the spacer, further defining the air inlet conduit.

The liquid barrier wall 20 couples to the spacer 21 by means of pegs 31 on the spacer engaging with holes in the underside of the liquid barrier wall 20. Similar to the dowels 53 in the lower body portion 52, the pegs 31 may engage with their respective holes by means of a clearance, transitional, or interference fit, and serve to prevent excessive movement of the liquid barrier wall 20 relative to the spacer 21.

The second portion of the capillary 24 is held in place within the indentations 37 in the spacer 21. Further, the slot 32 in the spacer 21, and both the straight 39 and annular portions 40 of the channel 38 in the manifold 22 are provided with a closing side, the air inlet conduit thereby defined.

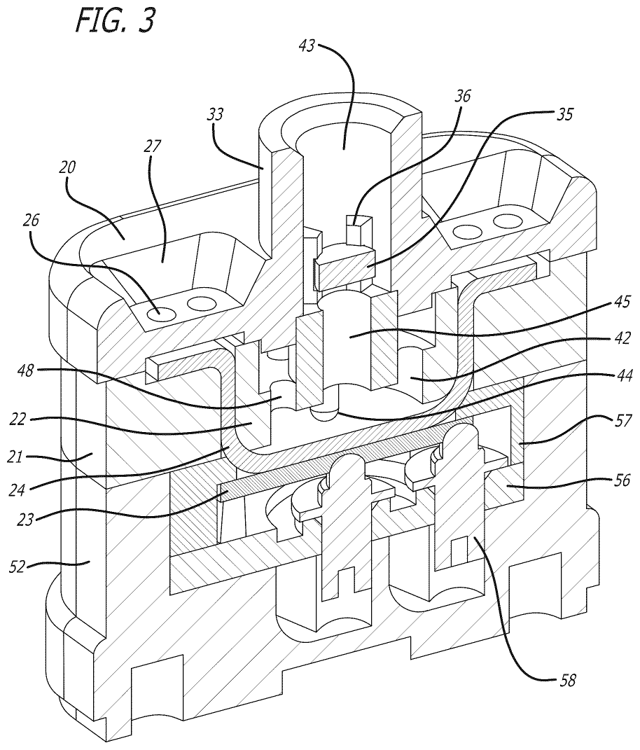

The lower body portion 52, the transducer 23 and transducer holder 50, the spacer 21, the capillary 24, the manifold 22 and the liquid barrier wall 20 form the subassembly illustrated in FIG. 3. The various slots and indentations interacting with the capillary 24 may be shaped and/or sized in order to compress the capillary 24 a predetermined amount. The compressive force on the capillary 24 is low enough to avoid restricting liquid transfer, but high enough to stop the liquid flooding the sonication chamber 42.

FIG. 3 further shows a possible configuration of the mist diverter assembly 34, should one be present. The plate 35 of the mist diverter assembly 34 is sized such that its diameter is at least the same as the bore of the aperture 45 in the manifold 22. The diverter therefore causes the mist to take a non-linear path through the mist outlet conduit, such a path not passable by liquid droplets larger than a predetermined size. In preferred examples, the plate 35 is of a larger diameter than the aperture 45. The splines 36 are preferably sized so as to not intrude on the diameter of the aperture 45, and thereby have minimal effect on the mist flow characteristics. An axial end of the splines 36 may advantageously act as a stop for the manifold 22 to ensure correct assembly.

The abovementioned subassembly carries the advantage of being simple to manufacture, and also simple to assemble. For example, at least some of the various holes, channels, and protrusions are two dimensional forms, and not intricate and complex geometries. The various sections are thus efficient to manufacture using well established manufacturing techniques, such as machining, casting, and moulding. This also means that manufacture and assembly may be at least partially autonomous. The parts of the device may be assembled using automated robots on a production line with minimal human intervention. The device is therefore configured to be mass produced on a production line relatively easily and at low cost compared with conventional mist generator devices.

The subassembly may be positioned within the housing 11 of the pod 10 such that the first section of the mist outlet conduit 46 is inserted into the central through hole 43 in the liquid barrier wall 20. The inner edge of the central protrusion 33 may be chamfered in order to aid insertion. Where present, the splines 36 may further act as a stop against the first portion 46 of the mist outlet conduit.

The second end wall 19 is coupled to the lower body portion 52 to close the assembly at the second axial end. The second end wall 19 may include a plurality of holes to provide continuity of the holes in the lower body portion 52. For example, the second end wall 19 may include an air inlet hole configured to align with the through hole 54 which forms a portion of the air inlet conduit.

The absorbing element 28 and the mouthpiece 12 are coupled at the first axial end of the housing 11 to form the pod 10 of FIG. 2.

The above-described pod 10 assembly comprises both an air-tight air inlet conduit and an air-tight mist outlet conduit, each formed of multiple components of the pod 10. Air may be conducted to the sonication chamber 42 from proximate the second end wall 19 via the through hole 54 in the lower body portion 52, the slot 32 formed in the spacer 21, the channel formed in the manifold 22, and through the one or more inlet apertures 48. After combining with the liquid particles, the mist exits the pod 10 through the first aperture 45 in the manifold 22, the mist diverter assembly 34 (where present), the first portion of the mist outlet conduit 46, the absorbent element 28, and the mouthpiece outlet port 17. The mist outlet conduit preferably passes through the liquid chamber 25.

Although the assembly has been described in a certain order, it will be appreciated that this is only an example and the components may be assembled in any plausible order. Similarly, terms such as “upper”, “lower”, and “side” are not to be construed as limiting, but for ease of reference to the figures.

The above-described pod 10 is configured to be coupled to a driver 59, the driver 59 comprising the means for powering and controlling the pod 10. The pod 10 is typically at least partially received by an axial end of the driver 59, and more specifically a cavity in the driver 59.

The driver 59 may house an electrical storage device configured to power the pod 10 so that the driver 59 generates a drive signal which drives an ultrasonic transducer 23 within the pod 10. The electrical storage device can be a battery, including but not limited to a lithium-ion, alkaline, zinc-carbon, nickel-metal hydride, or nickel-cadmium battery; a super capacitor; or a combination thereof. The electrical storage device may be rechargeable. In examples utilizing a rechargeable electrical storage device, a charging port may be provided so that the electrical storage device does not need to be removed from the driver 59. The electrical storage device may be primarily selected to deliver a constant voltage independent of charge level. Otherwise, the performance may degrade over time. Preferred electrical storage devices that are able to provide a consistent voltage output over the life of the device include lithium-ion and lithium polymer batteries.

Electrical communication between the driver 59 and the pod 10 may be established using electrical contacts 58.

A circuit board may carry at least one integrated circuit and/or a microprocessor. In some arrangements, the at least one integrated circuit and/or the microprocessor is configured to process data from a sensor which senses a parameter indicative of the operation of the ultrasonic transducer 23 and controls the driver 59 to vary the drive signal output to the ultrasonic transducer 23 in a feedback loop.

In some arrangements, the pod 10 or driver 59 comprises an activation sensor which detects when the user draws on the mouthpiece 12 and activates the ultrasonic transducer 23 to generate a mist. The activation sensor can be selected to detect changes in pressure, air flow, or vibration. In one arrangement, the activation sensor is a pressure sensor.

In some arrangements, the integrated circuit comprises a frequency controller which is configured to control the frequency of the drive signal output from the driver 59 to the ultrasonic transducer 23. The frequency controller comprises a processor and a memory, the memory storing executable instructions which, when executed by the processor, cause the processor to perform at least one function of the frequency controller.

In some arrangements, the driver 59 drives the ultrasonic transducer 23 with a signal having a frequency of 2.8 MHz to 3.2 MHz in order to atomize a liquid having a liquid viscosity of 1.05 Pa·s to 1.412 Pa·s. Such a frequency enables the ultrasonic transducer 23 to produce a bubble volume of about 0.25 to 0.5 microns. However, for liquids with a different viscosity or for other applications the ultrasonic transducer 23 may be driven at a different frequency. Parameters affecting the optimal frequency include the transducer manufacturing process and tolerances, the physical load on the transducer, the local and ambient temperature, and the distance from the transducer to the power source.

The driver 59 may have a wireless communication system, such as in the form of a Bluetooth Low Energy capable microcontroller. The wireless communication system is in communication with the at least one integrated circuit and/or the microprocessor of the device and is configured to transmit and receive data between the driver 59 and a computing device, such as a smartphone.

The connectivity via Bluetooth Low Energy to a companion mobile application allows for remote control of the mist inhalation device.

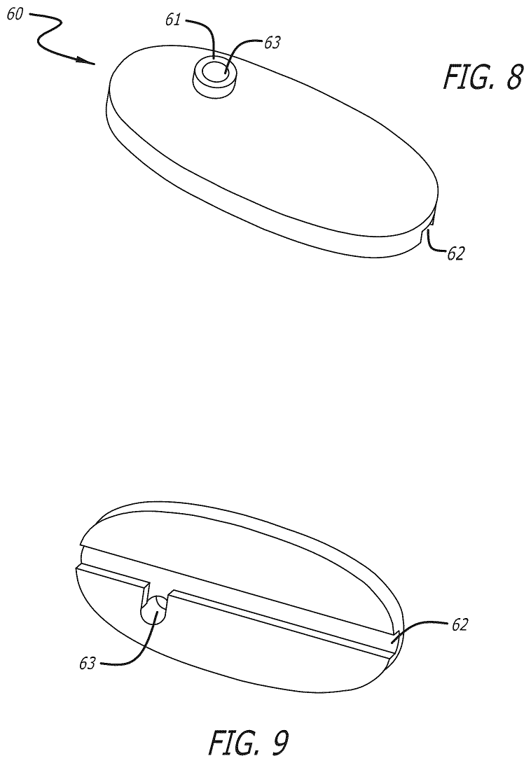

The base of the cavity in the driver 59 may house an insert 60 as shown in FIGS. 8 and 9. The insert may have a first, upper, surface; a second, lower, surface; and at least one side wall extending therebetween. The upper side of the insert 60 has a generally planar surface, and may include a protrusion 61. The protrusion 61 is sized and positioned to align with the air inlet hole in the second end wall 19 of the pod 10. In some examples, the protrusion 61 may not be present.

The lower side of the insert 60 may comprise a channel 62 which extends across the face of the lower side, as can be seen in FIG. 9. Alternatively, there may be provided a duct contained within the insert 60, the duct being enclosed by and passing through the first and second sides of the insert 60. The duct may include a port in the side wall of the insert 60. A hole 63 extends from the protrusion 61 to the channel 62 or duct, and thereby forms a section of the air flow path to the pod 10 from the driver 59. The channel 62 or duct may be of any diameter. A small diameter may be chosen, for example, to resist the user's inhalation and provide an improved user experience.

The insert 60 may be of a resiliently deformable material, preferably silicone, to effectively seal the connection between the protrusion 61 and the base of the pod 10.

Air from the surroundings may therefore enter the pod 10 via the driver 59 and optionally, the insert 60. FIG. 10 illustrates the airflow of some embodiments.

The air from the surroundings may enter the driver 59 in one of a number of ways. Example configurations will now be described with reference to FIGS. 11 to 14.

FIG. 11 shows a first configuration of the driver 59. In this example, the driver 59 includes two air intake holes 64 which pass through the driver housing wall, preferably transverse to or perpendicular to the major axis of the driver 59. The holes 64 align with the duct or channel 62 in the insert 60 such that an air flow path is created between the surroundings and the base of the pod 10. The holes 64 may be of any diameter and any number of holes 64 may be provided, the number of holes 64 preferably conforming to the number of holes and/or channels 62 in the insert 60. In some examples, the pod 10 comprises only one air intake hole 64, the intake hole 64 aligning with the duct or channel 62 in the insert 60. In such an example, the duct or channel 62 in the insert 60 may not extend from one side wall to the other as in FIG. 9; the duct or channel 62 may instead extend only from the hole 64 in the driver housing to the hole 63 in the insert 60.

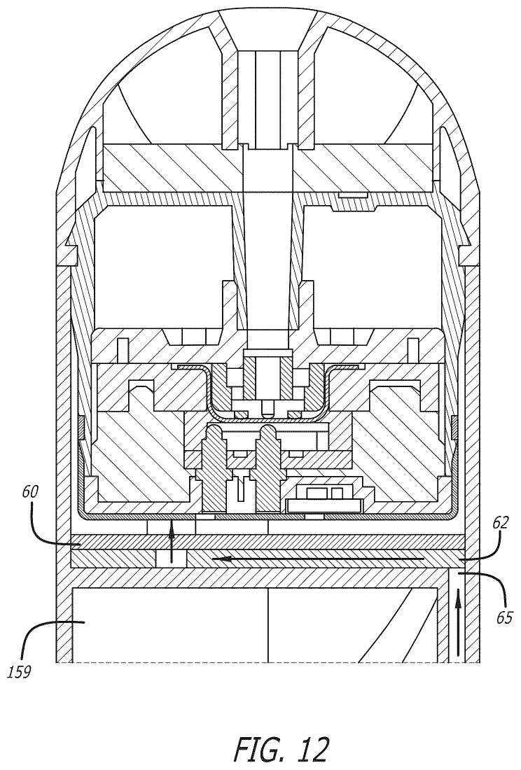

An alternative example of a driver 159 is shown in FIG. 12. An air inlet channel 65 is provided in the driver 159. At least a majority of the air inlet channel 65 extends substantially parallel to the major axis of the driver 159. Preferably, the inlet port (not shown) of the air inlet channel 65 is positioned at or towards the axial end of the driver 159 opposite the cavity for the pod 10. In other examples, the inlet port may be positioned in the side wall of the driver 159. The air inlet channel 65 may be of any diameter and any length, the parameters being selected based on factors such as user experience, efficiency, and packaging requirements. Further, more than one air inlet channel 65 may be provided. The air inlet channel 65 extends from the surroundings to the insert 60, and forms the air flow path to the base of the pod 10.

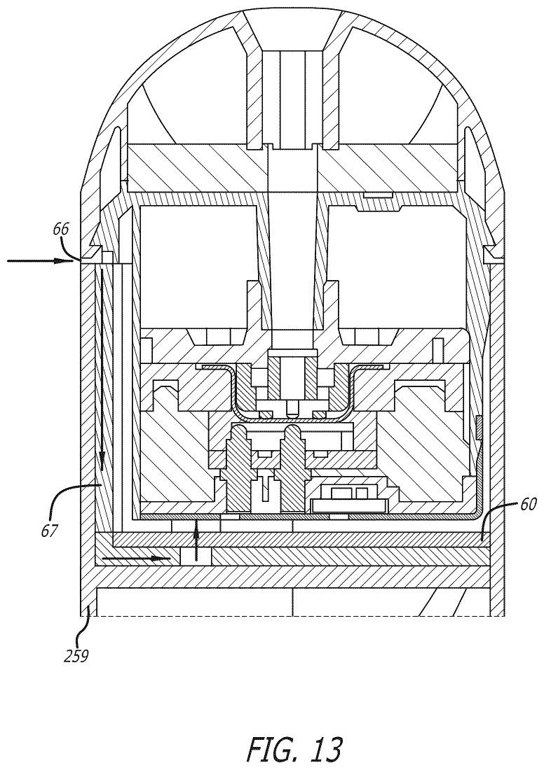

A further alternative example is shown in FIG. 13. In this example, at least one gap 66 is formed between the driver 259 and the mouthpiece 12 of the pod 10. An internal driver channel 67 is provided which extends parallel to the major axis of the driver 259. In this example, the driver channel 67 is formed as part of the driver 259, and the interior wall of the driver channel 67 defines a part of the cavity which is configured to accept the pod 10.

The duct or channel 62 in the insert 60 is adapted to accept air flow from the driver channel 67, and therefore provides an air flow path from the surroundings to the base of the pod 10.

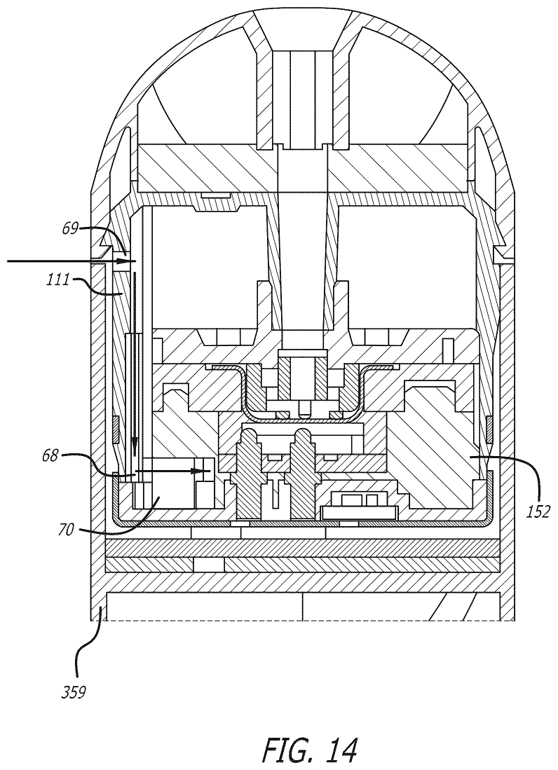

In the example illustrated in FIG. 14, a pod internal channel 68 is provided within the pod housing 111. The pod housing 111 further includes a hole 69 positioned towards a first end of the pod channel 68. Similar to the example of FIG. 13, a gap 66 is formed between the driver 359 and the mouthpiece 14 of the pod 110. Air from the surroundings may therefore enter the pod channel 68 via the gap 66 between the driver 359 and the mouthpiece 12, and the hole 69 in the pod housing 111.

A cut out 70 in the lower body portion 152 allows the air flow path to remain within the pod 110, i.e., without the need of the insert 60. The insert 60 may, however, remain part of the overall assembly.

It will be appreciated that further examples may fall within the scope of the appended claims, and that the illustrated examples should in no way be interpreted as limiting. For example, the examples of FIGS. 13 and 14 could be combined such that the internal driver channel 67 extends only part way into the cavity, and then aligns with a hole in the housing of the pod 10 and a cut out in the lower body portion 152.

The apparatus described above may comprise an identification arrangement that allows only genuine pods 10 from the manufacturer to be used with the driver 59. This anti-counterfeiting measure may be implemented in the pod 10 as a specific custom integrated circuit (IC) that is bonded to the pod 10. The IC contains truly unique information that allows complete traceability of the pod 10 (and its contents) over its lifetime as well as a precise monitoring of the consumption by the user. The IC allows the pod 10 to function and to generate mist only when authorized. The unique information can be read by the mist inhalation device to ascertain information such as whether the pod 10 is a genuine and/or certified pod, and whether the pod has been previously fully discharged, and therefore possibly refilled with counterfeit liquid. If certain conditions are met or not met, then the at least one integrated circuit and/or the microprocessor of the driver 59 may allow or prevent the use of the pod 10 with the driver 59.

It will be appreciated that the pod may be used in mist inhalation devices different to those disclosed, and vice versa. In such examples, the air inlet seal and the mist outlet seal are ruptured, the capillary conducts the liquid from the liquid chamber to the atomisation surface to be atomised at the atomisation surface, and the mist generated is conducted through the mist outlet conduit to the mist outlet port.

When used in this specification and the appended claims, the terms “comprises” and “comprising” and variations thereof mean that the specified features, steps or integers are included. The terms are not to be interpreted to exclude the presence of other features, steps or components.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

Protection may be sought for any features disclosed in any one or more published documents referenced herein in combination with the present disclosure.

Although certain example embodiments of the invention have been described, the scope of the appended claims is not intended to be limited solely to these embodiments. The claims are to be construed literally, purposively, and/or to encompass equivalents.

1. A mist inhalation pod for use with a driver and for delivering a therapeutic, the pod comprising:
a housing having a first end, an opposite second end and at least one side wall extending between the first end and the second end;
a first end wall proximate the first end and the side wall, the first end wall closing the first end of the housing, the first end wall being provided with a mist outlet port;
a second end wall proximate the second end and the side wall, the second end wall closing the second end of the housing;
a liquid barrier wall positioned within the housing and spaced apart from the first end wall, the liquid barrier wall extending towards the side wall of the housing to form a liquid seal between the liquid barrier wall and the side wall, the liquid barrier wall having at least one liquid channel with a liquid inlet and a liquid outlet;
a liquid chamber defined by the liquid barrier wall, the first end wall and the side wall of the housing, the liquid chamber containing a liquid to be atomised, the liquid comprising a therapeutic, the liquid inlet being in liquid communication with the liquid chamber for conducting the liquid through the liquid outlet;
a spacer positioned within the housing between the liquid barrier wall and the second end wall, the spacer having a perimeter extending toward the side wall of the housing, the spacer including a hollow interior surrounded by the perimeter;
a fluid flow manifold positioned at least partially within the hollow interior of the spacer and including a first side proximate the first end wall, and a second side proximate the second end wall, the first side of the fluid flow manifold including a channel, and the second side of the fluid flow manifold having a cavity, the cavity including a first aperture for allowing fluid flow in a first direction, and one or more further apertures for allowing fluid flow in a second direction;
a sonication chamber including the cavity of the fluid flow manifold;
an ultrasonic transducer positioned between the sonication chamber and the second end wall, the ultrasonic transducer having an atomisation surface adjacent to the sonication chamber and in communication with the sonication chamber;
a capillary having a first portion at least partly superimposed on the atomisation surface of the ultrasonic transducer and a second portion adjacent to the liquid outlet of the liquid channel, wherein the second portion of the capillary covers at least a portion of the liquid outlet and is configured to conduct the liquid from the liquid outlet to the atomisation surface to generate a mist;
an air inlet conduit, including the one or more further apertures in the fluid flow manifold, forming an air-tight channel for conducting air through the spacer and along the channel in the first side of the fluid flow manifold, the air inlet conduit extending from proximate the second end wall, through the spacer and through the fluid flow manifold to the sonication chamber, a first end of the air inlet conduit being in fluid communication with an air inlet port proximate the second end wall of the housing and a second end of the air inlet conduit being in fluid communication with the sonication chamber; and
a mist outlet conduit, including the first aperture in the fluid flow manifold, forming an air-tight channel for conducting the mist through the liquid chamber and any liquid contained therein, the mist outlet conduit extending from the first end wall, through the liquid chamber, through the liquid barrier wall, and through the fluid flow manifold to the sonication chamber, a first end of the mist outlet conduit being in fluid communication with a mist outlet port in the first end wall of the housing and a second end of the mist outlet conduit being in fluid communication with the sonication chamber,
wherein, in use, the capillary conducts the liquid from the liquid chamber to the atomisation surface to be atomised, and the mist generated is conducted through the mist outlet conduit to the mist outlet port.

2. The mist inhalation pod of claim 1, further comprising a mouthpiece positioned proximate the first end of the housing, the mouthpiece including a mist inhalation port in fluid communication with the mist outlet port in the housing.

3. The mist inhalation pod of claim 1, wherein the one or more further apertures include at least three apertures, the at least three apertures being spaced around the first aperture, the fluid flow in the first direction thereby being coaxial to the fluid flow in the second direction.

4. The mist inhalation pod of claim 1, wherein the one or more further apertures of the air inlet conduit are positioned radially inward of the first portion of the capillary, and air enters the sonication chamber transverse to the atomisation surface of the ultrasonic transducer.

5. The mist inhalation pod of claim 4, wherein the air enters the sonication chamber perpendicular to the atomisation surface of the ultrasonic transducer.

6. The mist inhalation pod of claim 1, wherein the liquid barrier wall has a first surface defining the liquid chamber, and a second surface coupled to the spacer, the first surface including a recess, and the liquid inlet of the liquid channel being positioned within the recess.

7. The mist inhalation pod of claim 1, wherein the first portion of the capillary substantially covers the atomisation surface of the ultrasonic transducer.

8. The mist inhalation pod of claim 1, wherein the mist outlet conduit includes a diverter, the diverter causing the mist to take a path through the mist outlet conduit not passable by liquid droplets larger than a predetermined size.

9. The mist inhalation pod of claim 8, wherein the mist outlet conduit has a first section and a second section, the first section having an internal diameter smaller than the internal diameter of the second section, and wherein the diverter comprises a plate having an internal diameter larger than the internal diameter of the first section of the mist outlet conduit and smaller than the internal diameter of the second section of the mist outlet conduit, such that the mist conducted from the sonication chamber travels along a non-linear path from the sonication chamber to the mist outlet port.

10. The mist inhalation pod of claim 9, wherein the diverter further includes a plurality of splines spaced around the internal diameter of the second section of the mist outlet conduit, the splines configured to support the plate.

11. The mist inhalation pod of claim 8, wherein the first section of the mist outlet conduit is the first aperture of the fluid flow manifold, and the second section of the mist outlet conduit is an aperture in the liquid barrier wall, the diverter being positioned within the second section of the mist outlet conduit.

12. A mist inhalation device comprising a pod for delivering a therapeutic, and a driver, the pod including:
a housing having a first end, an opposite second end and at least one side wall extending between the first end and the second end;
a first end wall proximate the first end and the side wall, the first end wall closing the first end of the housing, the first end wall being provided with a mist outlet port;
a second end wall proximate the second end and the side wall, the second end wall closing the second end of the housing;
a liquid barrier wall positioned within the housing and spaced apart from the first end wall, the liquid barrier wall extending towards the side wall of the housing to form a liquid seal between the liquid barrier wall and the side wall, the liquid barrier wall having at least one liquid channel with a liquid inlet and a liquid outlet;
a liquid chamber defined by the liquid barrier wall, the first end wall and the side wall of the housing, the liquid chamber containing a liquid to be atomised, the liquid comprising a therapeutic, the liquid inlet being in liquid communication with the liquid chamber for conducting the liquid through the liquid outlet;
a spacer positioned within the housing between the liquid barrier wall and the second end wall, the spacer having a perimeter extending toward the side wall of the housing, the spacer including a hollow interior surrounded by the perimeter;
a fluid flow manifold positioned at least partially within the hollow interior of the spacer and including a first side proximate the first end wall, and a second side proximate the second end wall, the first side of the fluid flow manifold including a channel, and the second side of the fluid flow manifold having a cavity, the cavity including a first aperture for allowing fluid flow in a first direction, and one or more further apertures for allowing fluid flow in a second direction;
a sonication chamber including the cavity of the fluid flow manifold;
an ultrasonic transducer positioned between the sonication chamber and the second end wall, the ultrasonic transducer having an atomisation surface adjacent to the sonication chamber and in communication with the sonication chamber;
a capillary having a first portion at least partly superimposed on the atomisation surface of the ultrasonic transducer and a second portion adjacent to the liquid outlet of the liquid channel, wherein the second portion of the capillary covers at least a portion of the liquid outlet and is configured to conduct the liquid from the liquid outlet to the atomisation surface to generate a mist;
an air inlet conduit, including the one or more further apertures in the fluid flow manifold, forming an air-tight channel for conducting air through the spacer and along the channel in the first side of the fluid flow manifold, the air inlet conduit extending from proximate the second end wall, through the spacer and through the fluid flow manifold to the sonication chamber, a first end of the air inlet conduit being in fluid communication with an air inlet port proximate the second end wall of the housing and a second end of the air inlet conduit being in fluid communication with the sonication chamber; and
a mist outlet conduit, including the first aperture in the fluid flow manifold, forming an air-tight channel for conducting the mist through the liquid chamber and any liquid contained therein, the mist outlet conduit extending from the first end wall, through the liquid chamber, through the liquid barrier wall, and through the fluid flow manifold to the sonication chamber, a first end of the mist outlet conduit being in fluid communication with a mist outlet port in the first end wall of the housing and a second end of the mist outlet conduit being in fluid communication with the sonication chamber,
wherein, in use, the capillary conducts the liquid from the liquid chamber to the atomisation surface to be atomised, and the mist generated is conducted through the mist outlet conduit to the mist outlet port, and
the driver including:
a housing having a cavity configured to receive at least part of the pod; and
driver circuitry for supplying a drive signal to the pod.

13. The mist inhalation device of claim 12, wherein the one or more further apertures include at least three apertures, the at least three apertures being spaced around the first aperture, the fluid flow in the first direction thereby being coaxial to the fluid flow in the second direction.

14. The mist inhalation device of claim 12, wherein the one or more further apertures of the air inlet conduit are positioned radially inward of the first portion of the capillary, and air enters the sonication chamber transverse to the atomisation surface of the ultrasonic transducer.

15. The mist inhalation device of claim 12, wherein the liquid barrier wall has a first surface defining the liquid chamber, and a second surface coupled to the spacer, the first surface including a recess, and the liquid inlet of the liquid channel being positioned within the recess.

16. The mist inhalation device of claim 12, wherein the first portion of the capillary substantially covers the atomisation surface of the ultrasonic transducer.

17. The mist inhalation device of claim 12, wherein the mist outlet conduit includes a diverter, the diverter causing the mist to take a path through the mist outlet conduit not passable by liquid droplets larger than a predetermined size.

18. The mist inhalation device of claim 17, wherein the mist outlet conduit has a first section and a second section, the first section having an internal diameter smaller than the internal diameter of the second section, and wherein the diverter comprises a plate having an internal diameter larger than the internal diameter of the first section of the mist outlet conduit and smaller than the internal diameter of the second section of the mist outlet conduit, such that the mist conducted from the sonication chamber travels along a non-linear path from the sonication chamber to the mist outlet port.

19. The mist inhalation device of claim 18, wherein the diverter further includes a plurality of splines spaced around the internal diameter of the second section of the mist outlet conduit, the splines configured to support the plate.

20. A mist inhalation device comprising a pod for delivering a therapeutic, and a driver, the pod including:
a housing having a first end, an opposite second end and at least one side wall extending between the first end and the second end;
a first end wall proximate the first end and the side wall, the first end wall closing the first end of the housing, the first end wall being provided with a mist outlet port;
a second end wall proximate the second end and the side wall, the second end wall closing the second end of the housing;
a liquid barrier wall positioned within the housing and spaced apart from the first end wall, the liquid barrier wall extending towards the side wall of the housing to form a liquid seal between the liquid barrier wall and the side wall, the liquid barrier wall having at least one liquid channel with a liquid inlet and a liquid outlet;
a liquid chamber defined by the liquid barrier wall, the first end wall and the side wall of the housing, the liquid chamber containing a liquid to be atomized, the liquid comprising a therapeutic, the liquid inlet being in liquid communication with the liquid chamber for conducting the liquid through the liquid outlet;
a spacer positioned within the housing between the liquid barrier wall and the second end wall, the spacer having a perimeter extending toward the side wall of the housing, the spacer including a hollow interior surrounded by the perimeter;
a fluid flow manifold positioned at least partially within the hollow interior of the spacer and including a first side proximate the first end wall, and a second side proximate the second end wall, the first side of the fluid flow manifold including a channel, and the second side of the fluid flow manifold having a cavity, the cavity including a first aperture for allowing fluid flow in a first direction, and one or more further apertures for allowing fluid flow in a second direction;
a sonication chamber including the cavity of the fluid flow manifold;
an ultrasonic transducer positioned between the sonication chamber and the second end wall, the ultrasonic transducer having an atomisation surface adjacent to the sonication chamber and in communication with the sonication chamber;
a capillary having a first portion at least partly superimposed on the atomisation surface of the ultrasonic transducer and a second portion adjacent to the liquid outlet of the liquid channel, wherein the second portion of the capillary covers at least a portion of the liquid outlet and is configured to conduct the liquid from the liquid outlet to the atomisation surface to generate a mist;
an air inlet conduit, including the one or more further apertures in the fluid flow manifold, forming an air-tight channel for conducting air through the spacer and along the channel in the first side of the fluid flow manifold, the air inlet conduit extending from proximate the second end wall, through the spacer and through the fluid flow manifold to the sonication chamber, a first end of the air inlet conduit being in fluid communication with an air inlet port proximate the second end wall of the housing and a second end of the air inlet conduit being in fluid communication with the sonication chamber; and
a mist outlet conduit, including the first aperture in the fluid flow manifold, forming an air-tight channel for conducting the mist through the liquid chamber and any liquid contained therein, the mist outlet conduit extending from the first end wall, through the liquid chamber, through the liquid barrier wall, and through the fluid flow manifold to the sonication chamber, a first end of the mist outlet conduit being in fluid communication with a mist outlet port in the first end wall of the housing and a second end of the mist outlet conduit being in fluid communication with the sonication chamber,
wherein, in use, the capillary conducts the liquid from the liquid chamber to the atomisation surface to be atomised, and the mist generated is conducted through the mist outlet conduit to the mist outlet port, and
the driver including:
a housing having at least one wall, and a cavity configured to receive at least a part of the pod;
an insert positioned within the cavity, the insert having a first surface, a second surface, and a side wall extending therebetween, the insert including a duct extending from the side wall to the first surface, the duct having an opening in the side wall and an opening in the first surface; and
a hole in the wall of the driver,
wherein the hole in the wall of the driver aligns with the opening in the side wall of the duct, and the opening in the first surface of the duct aligns with the air inlet conduit of the pod,
the air conducted from the surroundings to the air inlet conduit via the hole in the wall of the driver and the duct provided by the insert.
